(19) United States
(12) Patent Application Publication
(10) Pub. No.: US 2015/0336866 A1
(43) Pub. Date: Nov. 26, 2015

(54) SYNTHESIS OF DIFLUOROMETHYL ETHERS AND SULFIDES

(71) Applicant: THE REGENTS OF THE UNIVERSITY OF CALIFORNIA, Oakland, CA (US)

(72) Inventors: John HARTWIG, Berkeley, CA (US); Patrick FIER, Berkeley, CA (US)

(21) Appl. No.: 14/758,937

(22) PCT Filed: Dec. 23, 2013
(86) PCT No.: PCT/US13/77605

§ 371 Date: Jul. 1, 2015

Related U.S. Application Data
(60) Provisional application No. 61/748,119, filed on Jan. 1, 2013.

(51) Int. Cl. C07C 41/01; C07D 317/46; C07C 231/12; C07C 253/30; C07D 215/20; C07J 1/00; C07C 51/363; C07D 263/57; C07D 277/66; C07D 311/04; C07C 319/14; C07C 67/00; C07C 45/63

(52) U.S. Cl. CPC C07C 41/01; C07C 67/00; C07D 317/46; C07C 231/12; C07C 253/30; C07D 215/20; C07C 45/63; C07C 51/363; C07D 263/57; C07D 277/66; C07D 311/04; C07C 319/14; C07J 1/0059

(57) ABSTRACT

The synthesis of difluoromethyl ethers and sulfides with a simple, non-ozone-depleting reagent is described. The difluoromethylation of phenols with this reagent occurs at room temperature within minutes with exceptional functional group tolerance. The mild conditions makes possible tandem processes for the conversion of aryl boronic acids, aryl halides and arenes to difluoromethyl ethers. Mechanistic studies support a reaction pathway involving nucleophilic attack of the phenolate to difluorocarbene.

### CROSS-REFERENCE TO RELATED APPLICATIONS

This application claims under 35 USC 119(e) the benefit of U.S. Provisional Application No. 61/748,119, filed Jan. 1, 2013, which is incorporated herein by reference in its entirety for all purposes.

### STATEMENT AS TO RIGHTS TO INVENTIONS MADE UNDER FEDERALLY SPONSORED RESEARCH OR DEVELOPMENT

This invention was made with Government support under Grant No. GM-55382 awarded by the National Institutes of Health. The Government has certain rights in this invention.

### BACKGROUND OF THE INVENTION

Difluoromethyl ethers are found increasingly in pharmaceuticals, agrochemicals, and materials. [a) T. Hiyama, Organofluorine Compounds: Chemistry and Applications, Springer, Berlin; New York, 2000; b) P. Kirsch, Modern Fluoroorganic Chemistry: Synthesis, Reactivity, Applications, Wiley-VCH; Weinheim; Great Britain, 2004; c) J. B. Hu, W. Zhang, F. Wang, Chem. Commun. 2009, 7465-7478; d) P. Kirsch, M. Bremer, Angew. Chem. Int. Ed. 2000, 39, 4217-4235; e) J.-P. Bégué, D. Bonnet-Delpon, Bioorganic and Medicinal Chemistry of Fluorine, John Wiley & Sons, Hoboken, N.J., 2008.] Aryl difluoromethyl ethers are found in medicinally important compounds that include enzyme inhibitors, [N. Chauret, D. Guay, C. Li, S. Day, J. Silva, M. Blouin, Y. Ducharme, J. A. Yergey, D. A. Nicoll-Griffith, Bioorg. Med. Chem. Lett. 2002, 12, 2149-2152.] anti-HIV agents [T. Ohmine, T. Katsube, Y. Tsuzaki, M. Kazui, N. Kobayashi, T. Komai, M. Hagihara, T. Nishigaki, A. Iwamoto, T. Kimura, H. Kashiwase, M. Yamashita, Bioorg. Med. Chem. Lett. 2002, 12, 739-742.] and antimicrobial agents. [M. Takahata, J. Mitsuyama, Y. Yamashiro, M. Yonezawa, H. Araki, Y. Todo, S. Minami, Y. Watanabe, H. Narita, Antimicrobial Agents and Chemotherapy 1999, 43, 1077-1084.] Pantoprazole (Protonix®), a proton-pump inhibitor, is among the top 100 pharmaceuticals and contains a difluoromethyl ether. [S. M. Cheer, A. Prakash, D. Faulds, H. M. Lamb, Drugs 2003, 63, 101-132.]

However, current syntheses of difluoromethyl ethers require the ozone-depleting compound HCF₂Cl (Freon 22) that is difficult to handle because it is a gas (Scheme 1). [T. G. Miller, J. W. Thanassi, J. Org. Chem. 1960, 25, 2009-2012.] Non-ozone-depleting sources have been reported for the formation of difluoromethyl ethers from phenols, [a) Y. Zafrani, G. Sod-Moriah, Y. Segall, Tetrahedron 2009, 65, 5278-5283; b) L. J. Zhang, J. Zheng, J. B. Hu, J. Org. Chem. 2006, 71, 9845-9848; c) J. Zheng, Y. Li, L. J. Zhang, J. B. Hu, G. J. Meuzelaar, H. J. Federsel, Chem. Commun. 2007, 5149-5151; d) Q. Y. Chen, S. W. Wu, J Fluorine Chem 1989, 44, 433-440; e) Q. Y. Chen, S. W. Wu, J. Org. Chem. 1989, 54, 3023-3027.] but the reactions with these reagents often require high-temperatures, long reaction times, and have only been demonstrated to work with simple substrates.

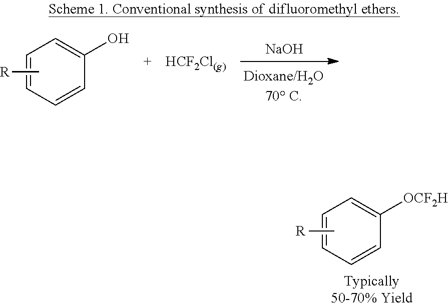

### SUMMARY OF THE INVENTION

The present invention provides methods and compositions for the difluoromethylation of aryl and vinyl compounds. In an exemplary method, the difluoromethylation is accomplished in a single step. The methods and compositions of the invention are highly versatile and are compatible with a wide range of substrates having a great variety of functional groups.

In contrast to the known syntheses of difluoromethyl ethers, the present invention provides compositions and methods for the operationally simple synthesis of difluoromethyl ethers and sulfides of broad scope with readily available reagents under mild conditions. The fast rates, tolerance for additional functionality and tolerance of byproducts formed by prior reactions make possible the development of one-pot protocols for the conversion of aryl halides, aryl boronic acids, and even arenes, to difluoromethyl ethers.

In an exemplary embodiment, the present invention provides a composition for forming a difluoromethyl ether or difluoromethyl sulfide. The composition comprises: (i) a precursor compound selected from an aryl alcohol, aryl thioalcohol and a vinylic alcohol, any of which are optionally further substituted; (ii) a difluoromethyl source; (iii) a base; and (iv) water.

Also provided are methods of preparing difluoromethyl ethers and difluoromethyl sulfides using a composition of the invention.

Additional objects, advantages and embodiments of the invention are set forth in the detailed description below.

### DETAILED DESCRIPTION OF THE EMBODIMENTS

### I. Introduction

The ability to selectively difluoromethylate an aryl or vinyl substrate has broad application, especially in the agricultural, pharmaceutical, and polymer industries. As described herein, the present invention relates to compositions and methods for transforming an aryl or vinyl substrate (such as an aryl alcohol, aryl thioalcohol, or vinylic alcohol) to the corresponding difluoromethyl ether or difluoromethyl sulfide. The compositions and methods of the invention utilize simple, readily available substrates and reaction mixtures and, thus, have wide applicability.

In various embodiments, the present invention provides a one-step procedure for the difluoromethylation of aryl and vinyl substrates (such as aryl alcohols, aryl thioalcohols, or vinylic alcohols) that occurs with readily available and non-hazardous reagents. This reaction tolerates a wide range of substituents, e.g., ester, amide, ketone, acetal, nitrile, aldehyde, and halogen functionalities, and occurs in high yield even with sterically hindered substrates. The simplicity and generality of this method makes it attractive for the introduction of a CF₂H group into functionally diverse arenes and vinyl compounds.

In various embodiments, the invention is directed to the aforementioned need in the art, and provides a new technique and compositions for effecting difluoromethylation of an aryl or vinyl precursor. The method involves contacting the precursor with a difluoromethyl source (e.g., difluoromethyltriflate) and an aqueous base.

Accordingly, the invention also provides a reaction mixture containing components of use to practice the method set forth above.

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are intended to be defined as indicated below.

### II. Definitions

Where substituent groups are specified by their conventional chemical formulae, written from left to right, the structures optionally also encompass the chemically identical substituents, which would result from writing the structure from right to left, e.g., —CH₂O— is intended to also optionally recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di-, tri- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to optionally include those derivatives of alkyl defined in more detail below, such as “heteroalkyl.” Alkyl groups that are limited to hydrocarbon groups are termed “homoalkyl”. Exemplary alkyl groups include the monounsaturated C₉₋₁₀, oleoyl chain or the diunsaturated C₉₋₁₀, ₁₂₋₁₃ linoeyl chain.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The terms “aryloxy” and “heteroaryloxy” are used in their conventional sense, and refer to those aryl or heteroaryl groups attached to the remainder of the molecule via an oxygen atom.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Further exemplary cycloalkyl groups include steroids, e.g., cholesterol and its derivatives. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to optionally include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: H, substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). These terms encompass groups considered exemplary “alkyl group substituents”, which are components of exemplary “substituted alkyl” and “substituted heteroalkyl” moieties.

Similar to the substituents described for the alkyl radical, substituents for the aryl and heteroaryl groups are generically referred to as “aryl group substituents.” The substituents are selected from, for example: H, substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′-C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl. These terms encompass groups considered exemplary “aryl group substituents”, which are components of exemplary “substituted aryl” and “substituted heteroaryl” moieties.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non-aromatic rings. Examples of “fused ring systems” are naphthalenes, indoles, quinolines, chromenes and the like.

As used herein, the term “heteroatom” includes oxygen (O), nitrogen (N), sulfur (S) and silicon (Si) and boron (B).

The symbol “R” is a general abbreviation that represents a substituent group that is selected from H, substituted or unsubstituted alkyl, substituted or unsubstituted heteroalkyl, substituted or unsubstituted aryl, substituted or unsubstituted heteroaryl, and substituted or unsubstituted heterocycloalkyl groups.

The terms “substrate” and “precursor” are used interchangeably and refer to compound with a leaving group substitutable by a difluoromethyl synthon in a method and composition of the invention. An exemplary substrate or precursor is an iodo-substituted aryl or vinyl compound which can react under the conditions of the invention, to yield at least one product having a difluoromethyl moiety.

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, “electron neutral”, “electron donating” and “electron withdrawing” refer to the net electronic effect of substituents on an aryl nucleus. The concept underlying electron neutral, electron donating and electron withdrawing substituents (e.g., aryl group substituents) is well-understood in the art and has been so for many years. Frameworks such as the Crum Brown-Gibson Rule (J. Chem Soc. 61, 367 (1892)) and the Hammett Equation (Hammett, Louis P. J. Am. Chem. Soc. 59, 96 (1937)) are a useful guide for the selection of individual substituents and combinations of substituents having electron neutral or electron donating properties. The selection of substituted aryl groups functioning within the methods of the invention utilizing the Crum Brown-Gibson Rule and Hammett Rule is a component of the instant invention.

The term “ligand” has the meaning ordinarily ascribed to it in the art. An exemplary ligand includes at least one donor atom capable of binding to a metal or metal ion. Ligands can include sterically bulky species, such as substituted or unsubstituted aryl, substituted or unsubstituted heteroaryl and substituted or unsubstituted fused ring systems, secondary and tertiary alkyl groups and the like.

The term “salt(s)” includes salts of the compounds prepared by the neutralization of acids or bases, depending on the particular ligands or substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium salt, or a similar salt. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic, propionic, isobutyric, butyric, maleic, malic, malonic, benzoic, succinic, suberic, fumaric, lactic, mandelic, phthalic, benzenesulfonic, p-tolylsulfonic, citric, tartaric, methanesulfonic, and the like. Certain specific compounds of the present invention contain both basic and acidic functionalities that allow the compounds to be converted into either base or acid addition salts. Hydrates of the salts are also included.

The symbol [symbol] , displayed perpendicular to a bond, indicates the point at which the displayed moiety is attached to the remainder of the molecule.

Below are examples of specific embodiments for carrying out the present invention. The examples are offered for illustrative purposes only, and are not intended to limit the scope of the present invention in any way.

In some embodiments, the definition of terms used herein is according to IUPAC.

### III. The Compositions

In an exemplary embodiment, the present invention provides a composition comprising: (i) a precursor compound selected from an aryl alcohol, aryl thioalcohol and a vinylic alcohol, any of which are optionally further substituted; (ii) a difluoromethyl source; (iii) a base; and (iv) water. When the precursor compound includes a substituted aryl moiety, this moiety is substituted with one or more independently selected members selected from the “aryl group substituents” described herein. When the precursor compound includes a substituted vinyl moiety, this moiety is substituted with one or more independently selected members selected from the “alkyl group substituents” described herein.

The composition functions to transform aryl alcohols, aryl thioalcohols or vinylic alcohols of a broad range of structures to the corresponding difluoromethyl ethers or difluoromethyl sulfides. For example, in addition to the alcohol or thioalcohol moiety, the precursor compound is optionally further substituted (for example, with an ester, amide, ketone, acetal, nitrile, aldehyde, halogen, heterocycle or a combination thereof).

### IIIa. Precursor Compound

In some embodiments, the precursor compound is a member selected from a substituted or unsubstituted aryl alcohol, a substituted or unsubstituted aryl thioalcohol and a substituted or unsubstituted vinylic alcohol, any of which are optionally further substituted (in addition to the —OH or —SH moieties of the aryl alcohol and aryl thioalcohol, respectively, and the —OH moiety of the vinylic alcohol).

In some embodiments, the —OH or —SH moieties of the precursor compound is in deprotonated form (that is, —O⁻ or —S⁻, respectively).

In some embodiments, the precursor compound is an aryl alcohol (such as a phenol). In some embodiments, the precursor compound is an aryl thioalcohol (such as a thiophenol). In some embodiments, the precursor compound is a stable enol.

In some embodiments, the precursor compound has the formula:

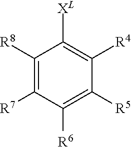

wherein R⁴, R⁵, R⁶, R⁷, and R⁸ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂, wherein two or more of R⁴, R⁵, R⁶, R⁷ and R⁸, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl. X^{L} is a member selected from —OH, —SH, —O⁻ and —S⁻.

The symbols R⁹ and R¹⁰ represent members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl.

In some embodiments, the precursor compound has the formula:

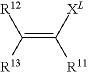

wherein R¹¹, R¹², and R¹³ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂. Two or more of R¹¹, R¹², and R¹³, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl. R⁹ and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring. X^{L} is a member selected from —OH, —SH, —O⁻ and —S⁻.

Examples of the diversity of precursor compounds of use in the compositions and methods of the invention are set forth in Example 1-4.

In some embodiments, the precursor compound is synthesized in situ. Exemplary in situ syntheses of the precursor compound are set forth in Examples 2-4.

### IIIb. Difluoromethyl Source

Any source of difluoromethyl is of use in the present invention. In some embodiments, the difluoromethyl source has the formula HCF₂OSO₂R^{x}, wherein R^{x} is fluoroalkyl or aryl.

In some embodiments, R^{x} is C₁, C₂, C₃, C₄, C₅ or C₆ fluoroalkyl in which the carbons are substituted with one, two or three fluoro moieites and when R^{x} includes more than one carbon atom, the substitution on each carbon atom is independently selected. In some embodiments, R^{x} is C₁, C₂, C₃, C₄, C₅ or C₆ perfluoroalkyl.

In some embodiments, R^{x} is substituted aryl. In some embodiments, R^{x} is aryl substituted with one, two, three, four or five members independently selected from fluorine and fluoroalkyl. In some embodiments, R^{x} is perfluoroaryl. In some embodiments, R^{x} is aryl substituted with one, two, three, four or five fluoroalkyl groups.

In some embodiments, the difluoromethyl source is difluoromethyltriflate (HCF₂Tf). In some embodiments, the difluoromethyl source is difluoromethylnonaflate (HCF₂Nf). In some embodiments, the difluoromethyl source is difluoromethyl perfluoroethylsulfonate, difluoromethyl perfluoropropylsulfonate, and difluoromethyl perfluorohexylsulfonate.

In some embodiments, the difluoromethyl source is non-ozone-depleting.

Difluoromethyltriflate is an attractive source of a difluoromethyl unit because it can be prepared in multi-gram scale from readily available, non-ozone-depleting reagents. The reaction between TMSCF₃ (the Ruppert-Prakash reagent) and triflic acid with catalytic TiCl₄ at room temperature provides difluoromethyltriflate (HCF₂OTf) in good yield (eq. 1). [V. V. Levin, A. D. Dilman, P. A. Belyakov, M. I. Struchkova, V. A. Tartakovsky, J. Fluor. Chem. 2009, 130, 667-670.] HCF₂OTf is an air-stable liquid which makes handling the reagent easier than gaseous HCF₂Cl.

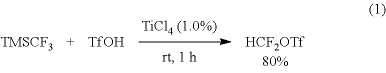

### IIIc. Base

In some embodiments, the base is a member selected from KOH, LiOH, NaOH, CsOH, LiN(SiMe₃)₂, NaN(SiMe₃)₂, KN(SiMe₃)₂, NaH, LiOtBu, NaOtBu, and KOtBu. In some embodiments, the base is a member selected from KOH, LiOH, NaOH. In some embodiments, the base is KOH.

### IIId. Co-Solvent

In some embodiments, the composition further comprises a co-solvent. The co-solvent can be any compound or mixture of compounds useful to dissolve at least a portion of one or more component of the composition. In some embodiments, the co-solvent is a polar organic solvent, such as DMF, DMSO, and MeCN (acetonitrile). In some embodiments, the co-solvent is water-miscible. In some embodiments, the co-solvent is a member selected from DMF, DMSO, dioxane, THF, and MeCN. In some embodiments, the co-solvent is MeCN.

### IIIe. Exemplary Compositions

Any combination of precursor compound, difluoromethyl source, base, water and, optionally, co-solvent is encompassed by this disclosure and specifically provided by the invention.

In an exemplary embodiment, the present invention provides a composition comprising:

(i) a precursor compound selected from an aryl alcohol, aryl thioalcohol and a vinylic alcohol, any of which are optionally further substituted;
(ii) a difluoromethyl source having the formula HCF₂X, wherein X has the formula —OSO₂R^{x}, wherein R^{x} is fluoroalkyl;
(iii) a base; and
(iv) water.

In some embodiments, the precursor compound is an aryl alcohol or an aryl thioalcohol, which is optionally further substituted. In some embodiments, the precursor compound is a phenol or a thiophenol, any of which are optionally further substituted. In some embodiments, the precursor compound is a phenol, which is optionally further substituted.

In some embodiments, the precursor compound is selected from an aryl alcohol, aryl thioalcohol and a vinylic alcohol, any of which are optionally further substituted; the difluoromethyl source is difluoromethyltriflate (HCF₂Tf) or difluoromethylnonaflate (HCF₂Nf); the base is KOH.; and the co-solvent is MeCN.

In some embodiments, the precursor compound is an aryl alcohol or an aryl thioalcohol, which is optionally further substituted. In some embodiments, the precursor compound is a phenol or a thiophenol, any of which are optionally further substituted. In some embodiments, the precursor compound is a phenol, which is optionally further substituted.

In some embodiments, the precursor compound is an aryl alcohol or aryl thioalcohol; and said precursor compound was synthesized in situ. In some embodiments, the aryl alcohol was synthesized in situ from an arylboronic acid, an aryl halide, or an arene. Exemplary in situ syntheses of the aryl alcohol from an arylboronic acid, an aryl halide (such as aryl bromide and aryl chloride), or an arene are set forth in Example 2, 3 and 4, respectively.

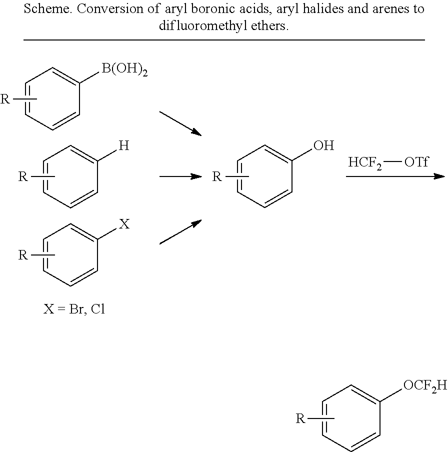

As shown in Table 1 (see Example 1), various ratios of precursor compound, difluoromethyl source, and base are of use in compositions of the invention. In an exemplary embodiment, the precursor compound, the difluoromethyl source, and the base are present in the composition in a molar ratio of about 1:3:12.

### IV. The Methods

In various embodiments, the present invention provides methods for converting an aryl alcohol, aryl thioalcohol or vinylic alcohol to the corresponding difluoromethyl ether or difluoromethyl sulfide. In an exemplary embodiment, the method includes: (a) forming a composition as set forth herein; and (b) incubating the composition under conditions appropriate to form the difluoromethyl ether or the difluoromethyl sulfide by difluoromethylating the alcohol or thioalcohol moiety of the precursor compound.

According to the method of the invention, any useful temperature or range of temperatures can be used to convert the precursor to the desired product. In an exemplary embodiment, the reaction mixture is incubated at a temperature from about 0° C. to about 40° C., e.g., about 20° C. (room temperature).

The reaction mixture can be incubated for any useful length of time. In various embodiments, the invention is incubated at a desired temperature for about 1 minute to about 24 hours, e.g., for about 1 minute to about 1 hour, e.g., for about 1 minute to about 30 minutes, e.g., for about 1 minute to about 10 minutes, e.g., for about 1 minute to about 5 minutes, e.g. for about 2 minutes.

The reaction mixture can be incubated in a vessel of any useful configuration. In an exemplary embodiment, the vessel is sealed while the reaction mixture is incubated, e.g., a sealed tube.

The following examples illustrate embodiments of the invention and are not intended to limit the scope of the compositions of the invention or the methods in which they find use.

### EXAMPLES

### General Experimental Details

All manipulations were conducted on the benchtop without any exclusion of air or moisture, unless otherwise noted. All reactions were conducted in 4 mL or 20 mL vials fitted with a Teflon-lined screw cap unless otherwise noted.

HCF₂OTf and HCF₂ONf were prepared according to the published procedure. [Levin, V. V.; Dilman, A. D.; Belyakov, P. A.; Struchkova, M. I.; Tartakovsky, V. A. J. Fluor. Chem. 2009, 130, 667.]. All other reagents were purchased from commercial suppliers and used as received.

NMR spectra were acquired on 400 MHz, 500 MHz, or 600 MHz Bruker instruments at the University of California, Berkeley. NMR spectra were processed with MestReNova 5.0 (Mestrelab Research SL). Chemical shifts are reported in ppm and referenced to residual solvent peaks (CHCl₃ in CDCl₃: 7.26 ppm for ¹H and 77.0 ppm for ¹³C) or to an external standard (1% CFCl₃ in CDCl₃: 0 ppm for ¹⁹F). Coupling constants are reported in hertz.

All GC-MS analyses were conducted with an Agilent 6890N GC equipped with an HP-5 column (25 m×0.20 mm ID×0.33 m film) and an Agilent 5973 Mass Selective Detector. The temperature for each run was held at 50° C. for 2 min, ramped from 50° C. to 300° C. at 40° C./min, and held at 300 OC for 5 min.

### Example 1

### Screen of Reaction Conditions for the Difluoromethylation of 4-Butylphenol with HCF₂OTf and KOH

TABLE 1
Screen of reaction conditions for the difluoromethylation of
4-butylphenol with HCF₂OTf and KOH.

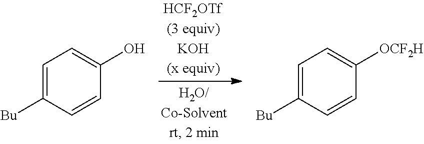

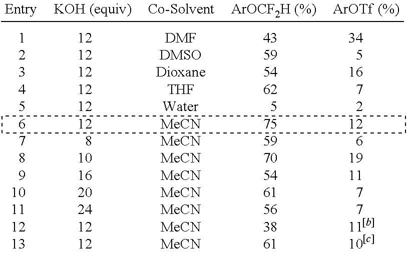

^{[a]}Reactions were performed on a 0.1 mmol scale and the yields were determined by GC with 1-bromo-4-fluorobenzene as an internal standard.
^{[b]}The reaction was performed with LiOH in place of KOH.
^{[c]}The reaction was performed with NaOH in place of KOH.

Difluoromethylation of Phenols with HCF₂OTf.

TABLE 2
Difluoromethylation of phenols with HCF₂OTf.

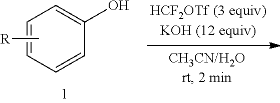

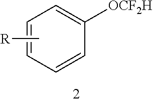

| | |
|---|---|
| 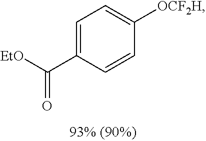 | 2a |
| 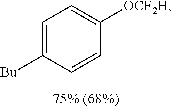 | 2b |
| 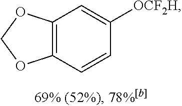 | 2c |
| 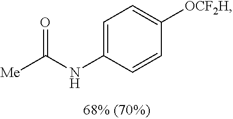 | 2d |
| 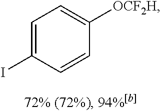 | 2e |
| 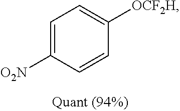 | 2f |
| 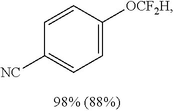 | 2g |
| 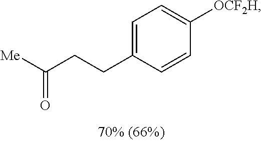 | 2h |
| 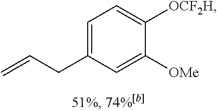 | 2i |
| 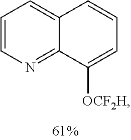 | 2j |
| 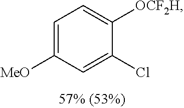 | 2k |
| 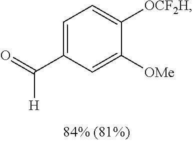 | 2l |
| 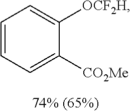 | 2m |
| 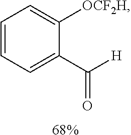 | 2n |
| 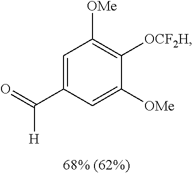 | 2o |
| 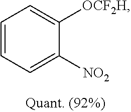 | 2p |
| 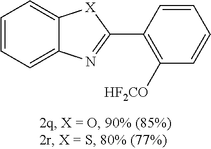 | |
| 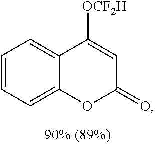 | 2s |
| 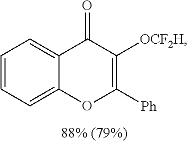 | 2t |
| 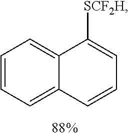 | 2u |
| 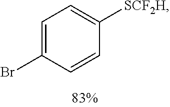 | 2v |
| 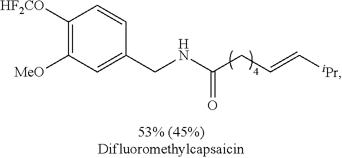 | 2w |
| 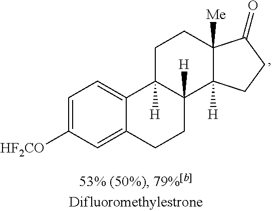 | 2x |

^{[a]}Reactions were performed on a 0.1 mmol scale to determine yields by ¹⁹F NMR spectroscopy with PhCF₃ as an internal standard. Isolated yields are shown in parenthesis for reactions performed on a 0.5 mmol scale.
^{[b]}Reactions were performed on a 0.1 mmol scale with HCF₂ONf in place of HCF₂OTf and yields were determined by ¹⁹F NMR spectroscopy.

In each reaction, the only by-products observed were unreacted phenol and varying amounts of aryl-triflate. [The aryl triflate is formed by nucleophilic attack of the phenoxide on the sulfur atom of HCF₂OTf.] The aryl triflate from reactions with HCF₂OTf can be recycled to the starting phenol by basic hydrolysis (eq 2). [T. Ohgiya, S. Nishiyama, Tet. Lett. 2004, 45, 6317-6320.]

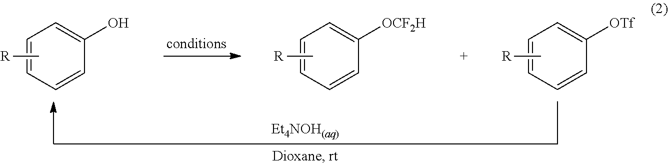

### General Procedure for the Difluoromethylation of Phenols and Thiophenols

Into a 20 mL vial was placed the phenol or thiophenols (0.5 mmol, 1.0 equiv), acetonitrile (1.0 mL) and 6M aqueous KOH (1.0 mL). The mixture was stirred rapidly at room temperature and HCF₂OTf (210 μL, 1.5 mmol, 3.0 equiv) was added at once. Note: the reactions are exothermic. The mixture was stirred vigorously for 2 minutes. The reaction was diluted with H₂O (8 mL) and extracted with ether (2×8 mL). The combined organic layers were dried over MgSO₄, concentrated, and purified by silica gel chromatography.

### Ethyl 4-(difluoromethoxy)benzoate (2a)

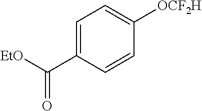

The reaction was performed according to the general procedure for the difluoromethylation of phenols on a 0.5 mmol scale. The product was purified by silica gel chromatography to give 2a as a clear oil (98 mg, 90% yield).

¹H NMR (500 MHz, CDCl₃) δ 8.06 (d, J=8.7 Hz, 2H), 7.15 (d, J=8.5 Hz, 2H), 6.59 (t, J=73.2 Hz, 1H), 4.37 (q, J=7.1 Hz, 2H), 1.39 (t, J=7.1 Hz, 3H).

¹³C NMR (151 MHz, CDCl₃) δ 165.66 (s), 154.61 (t, J=2.4 Hz), 131.60 (s), 127.47 (s), 118.59 (s), 115.40 (t, J=261.0 Hz), 61.13 (s), 14.29 (s).

¹⁹F NMR (376 MHz, CDCl₃) δ −84.25 (d, J=73.2 Hz).

### 1-butyl-4-(difluoromethoxy)benzene (2b)

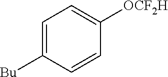

The reaction was performed according to the general procedure for the difluoromethylation of phenols on a 0.5 mmol scale. The product was purified by silica gel chromatography to give 2b as a clear oil (68 mg, 68% yield).

¹H NMR (600 MHz, CDCl₃) δ 7.16 (d, J=8.2 Hz, 2H), 7.03 (d, J=8.0 Hz, 2H), 6.47 (t, J=74.3 Hz, 1H), 2.59 (t, J=7.7 Hz, 2H), 1.62-1.54 (m, 2H), 1.35 (dd, J=14.8, 7.4 Hz, 2H), 0.93 (t, J=7.3 Hz, 3H).

¹³C NMR (151 MHz, CDCl₃) δ 149.19 (t, J=2.8 Hz), 140.17 (s), 129.61 (s), 119.45 (s), 116.15 (t, J=258.9 Hz), 34.89 (s), 33.61 (s), 22.25 (s), 13.89 (s).

¹⁹F NMR (376 MHz, CDCl₃) δ −82.61 (d, J=74.3 Hz).

### 5-(difluoromethoxy)benzo[d][1,3]dioxole (2c)

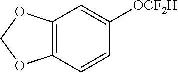

The reaction was performed according to the general procedure for the difluoromethylation of phenols on a 0.5 mmol scale. The product was purified by silica gel chromatography to give 2c as a clear oil (49 mg, 52% yield).

¹H NMR (600 MHz, CDCl₃) δ 6.75 (d, J=8.4 Hz, 1H), 6.67 (s, 1H), 6.59 (d, J=8.4 Hz, 1H), 6.40 (t, J=74.1 Hz, 1H), 5.99 (s, 2H).

¹³C NMR (151 MHz, CDCl₃) δ 148.27 (s), 145.47 (t, J=3.1 Hz), 145.32 (s), 116.19 (t, J=260.0 Hz), 112.72 (s), 108.07 (s), 102.80 (s), 101.78 (s).

¹⁹F NMR (376 MHz, CDCl₃) δ −82.74 (d, J=74.1 Hz).

### N-(4-(difluoromethoxy)phenyl)acetamide (2d)

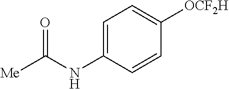

The reaction was performed according to the general procedure for the difluoromethylation of phenols on a 0.5 mmol scale. The product was purified by silica gel chromatography to give 2d as a white solid (70 mg, 70% yield).

¹H NMR (600 MHz, CDCl₃) δ 7.49 (d, J=8.7 Hz, 2H), 7.25 (br s, 1H), 7.08 (d, J=8.6 Hz, 2H), 6.46 (t, J=74.0 Hz, 1H), 2.18 (s, 3H).

¹³C NMR (151 MHz, CDCl₃) δ 168.36 (s), 147.31 (t, J=2.3 Hz), 135.32 (s), 121.32 (s), 120.40 (s), 115.94 (t, J=260.0 Hz), 24.42 (s).

¹⁹F NMR (376 MHz, CDCl₃) δ −82.52 (d, J=74.0 Hz).

### 1-(difluoromethoxy)-4-iodobenzene (2e)

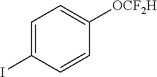

The reaction was performed according to the general procedure for the difluoromethylation of phenols on a 0.5 mmol scale. The product was purified by silica gel chromatography to give 2e as a clear oil (98 mg, 72% yield).

¹H NMR (400 MHz, CDCl₃) δ 7.67 (d, J=8.9 Hz, 2H), 6.89 (d, J=8.8 Hz, 2H), 6.48 (t, J=73.4 Hz, 1H).

¹³C NMR (151 MHz, CDCl₃) δ 150.89 (t, J=2.9 Hz), 138.82 (s), 121.80 (s), 115.54 (t, J=261.2 Hz), 89.08 (s).

¹⁹F NMR (376 MHz, CDCl₃) δ −83.62 (d, J=73.4 Hz).

### 1-(difluoromethoxy)-4-nitrobenzene (2)

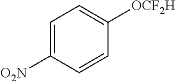

The reaction was performed according to the general procedure for the difluoromethylation of phenols on a 0.5 mmol scale. The product obtained from the aqueous workup as a white solid (2f) was not subjected to further purification (88 mg, 94% yield).

¹H NMR (600 MHz, CDCl₃) δ 8.27 (d, J=8.5 Hz, 2H), 7.25 (d, J=8.1 Hz, 2H), 6.63 (t, J=72.2 Hz, 1H).

¹³C NMR (151 MHz, CDCl₃) δ 155.52 (t, J=2.8 Hz), 144.81 (s), 125.75 (s), 119.34 (s), 114.99 (t, J=263.7 Hz).

¹⁹F NMR (376 MHz, CDCl₃) δ −85.19 (d, J=72.2 Hz).

### 4-(difluoromethoxy)benzonitrile (2g)

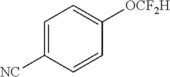

The reaction was performed according to the general procedure for the difluoromethylation of phenols on a 0.5 mmol scale. The product obtained from the aqueous workup as a white solid (2g) was not subjected to further purification (88 mg, 94% yield).

¹H NMR (600 MHz, CDCl₃) δ 7.69 (d, J=8.6 Hz, 2H), 7.22 (d, J=8.5 Hz, 2H), 6.59 (t, J=72.4 Hz, 1H).

¹³C NMR (151 MHz, CDCl₃) δ 154.09 (t, J=2.8 Hz), 134.14 (s), 119.84 (s), 118.00 (s), 115.05 (t, J=263.2 Hz), 109.17 (s).

¹⁹F NMR (376 MHz, CDCl₃) δ −84.85 (d, J=72.4 Hz).

### 4-(4-(difluoromethoxy)phen butan-2-one (2h)

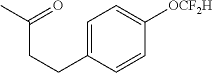

The reaction was performed according to the general procedure for the difluoromethylation of phenols on a 0.5 mmol scale. The product was purified by silica gel chromatography to give 2h as a clear oil (71 mg, 66% yield).

¹H NMR (600 MHz, CDCl₃) δ 7.17 (d, J=8.3 Hz, 2H), 7.03 (d, J=8.3 Hz, 2H), 6.47 (t, J=74.1 Hz, 1H), 2.88 (t, J=7.5 Hz, 2H), 2.75 (t, J=7.5 Hz, 2H), 2.14 (s, 3H).

¹³C NMR (151 MHz, CDCl₃) δ 207.48 (s), 149.49 (t, J=2.9 Hz), 138.29 (s), 129.62 (s), 119.70 (s), 116.00 (t, J=259.4 Hz), 44.98 (s), 30.04 (s), 28.88 (s).

¹⁹F NMR (376 MHz, CDCl₃) δ −82.71 (d, J=74.1 Hz).

### 2-chloro-1-(difluoromethoxy)-4-methoxybenzene (2k)

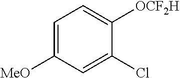

The reaction was performed according to the general procedure for the difluoromethylation of phenols on a 0.5 mmol scale. The product was purified by silica gel chromatography to give 2k (56 mg, 53% yield).

¹H NMR (600 MHz, CDCl₃) δ 7.17 (d, J=9.0 Hz, 1H), 6.97 (d, J=3.0 Hz, 1H), 6.78 (dd, J=9.0, 3.0 Hz, 1H), 6.44 (t, J=74.0 Hz, 1H), 3.79 (s, 3H).

¹³C NMR (151 MHz, CDCl₃) δ 157.69 (s), 140.31 (t, J=3.1 Hz), 127.38 (s), 123.54 (s), 116.04 (t, J=262.1 Hz), 115.69 (s), 113.40 (s), 55.80 (s).

¹⁹F NMR (376 MHz, CDCl₃) δ −83.09 (d, J=74.1 Hz).

### 4-(difluoromethoxy)-3-methoxybenzaldehyde (2l)

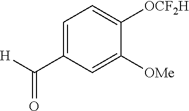

The reaction was performed according to the general procedure for the difluoromethylation of phenols on a 0.5 mmol scale. The product was purified by silica gel chromatography to give 2l as a white solid (81 mg, 81% yield).

¹H NMR (400 MHz, CDCl₃) δ 9.94 (s, 1H), 7.50 (s, 1H), 7.47 (d, J=8.1 Hz, 1H), 7.31 (d, J=8.1 Hz, 1H), 6.67 (t, J=74.2 Hz, 1H), 3.96 (s, 3H).

¹³C NMR (151 MHz, CDCl₃) δ 190.81 (s), 151.46 (s), 144.86 (t, J=2.6 Hz), 134.45 (s), 124.98 (s), 121.40 (s), 115.52 (t, J=261.6 Hz), 110.91 (s), 56.13 (s).

¹⁹F NMR (376 MHz, CDCl₃) δ −83.73 (d, J=74.2 Hz).

### Methyl 2-(difluoromethoxy)benzoate (2m)

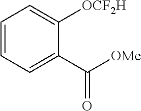

The reaction was performed according to the general procedure for the difluoromethylation of phenols on a 0.5 mmol scale. The product was purified by silica gel chromatography to give 2m (66 mg, 65% yield).

¹H NMR (600 MHz, CDCl₃) δ 7.90 (d, J=7.8 Hz, 1H), 7.54 (t, J=7.8 Hz, 1H), 7.32 (t, J=7.6 Hz, 1H), 7.26 (d, J=8.2 Hz, 1H), 6.57 (t, J=74.7 Hz, 1H), 3.92 (s, 3H).

¹³C NMR (151 MHz, CDCl₃) δ 165.50 (s), 149.89 (t, J=3.2 Hz), 133.55 (s), 131.70 (s), 125.96 (s), 124.35 (s), 122.92 (s), 116.43 (t, J=260.6 Hz), 52.37 (s).

¹⁹F NMR (376 MHz, CDCl₃) δ −83.47 (d, J=74.7 Hz).

### 1-(difluoromethoxy)-2-nitrobenzene (2o)

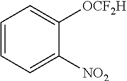

The reaction was performed according to the general procedure for the difluoromethylation of phenols on a 0.5 mmol scale. The product obtained from the aqueous workup as a clear oil (2o) was not subjected to further purification (87 mg, 92% yield).

¹H NMR (600 MHz, CDCl₃) δ 7.93 (d, J=8.0 Hz, 1H), 7.63 (t, J=7.9 Hz, 1H), 7.40 (m, 2H), 6.62 (t, J=73.0 Hz, 1H).

¹³C NMR (151 MHz, CDCl₃) δ 143.07 (t, J=3.2 Hz), 142.88 (s), 134.09 (s), 126.38 (s), 125.62 (s), 123.56 (s), 115.61 (t, J=265.2 Hz).

¹⁹F NMR (376 MHz, CDCl₃) δ −84.01 (d, J=73.0 Hz).

### 4-(difluoromethoxy)-3,5-dimethoxybenzaldehyde (2p)

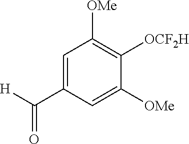

The reaction was performed according to the general procedure for the difluoromethylation of phenols on a 0.5 mmol scale. The product was purified by silica gel chromatography to give 2p as a white solid (72 mg, 62% yield).

¹H NMR (400 MHz, CDCl₃) δ 9.92 (s, 1H), 7.16 (s, 2H), 6.66 (t, J=75.9 Hz, 1H), 3.96 (s, 6H).

¹³C NMR (151 MHz, CDCl₃) δ 190.73 (s), 153.54 (s), 134.10 (s), 134.03 (t, J=2.8 Hz), 116.16 (t, J=261.4 Hz), 106.34 (s), 56.53 (s).

¹⁹F NMR (376 MHz, CDCl₃) δ −83.00 (d, J=75.9 Hz).

### 2-(2-(difluoromethoxy)phenyl)benzo[d]oxazole (2q)

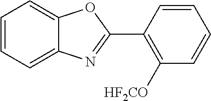

The reaction was performed according to the general procedure for the difluoromethylation of phenols on a 0.5 mmol scale. The product was purified by silica gel chromatography to give 2q as a white solid (111 mg, 85% yield).

¹H NMR (400 MHz, CDCl₃) δ 8.25 (d, J=7.7 Hz, 1H), 7.85-7.79 (m, 1H), 7.62 (dd, J=5.0, 3.9 Hz, 1H), 7.56 (td, J=8.2, 1.2 Hz, 1H), 7.45-7.36 (m, 4H), 6.75 (t, J=74.3 Hz, 1H).

¹³C NMR (151 MHz, CDCl₃) δ 160.05 (s), 150.63 (s), 149.02 (t, J=2.7 Hz), 141.74 (s), 132.57 (s), 131.24 (s), 126.25 (s), 125.51 (s), 124.64 (s), 122.76 (s), 120.58 (s), 120.43 (s), 116.24 (t, J=261.7 Hz), 110.69 (s).

¹⁹F NMR (376 MHz, CDCl₃) δ −83.59 (d, J=74.3 Hz).

### 2-(2-(difluoromethoxy)phenyl)benzo[d]thiazole (2r)

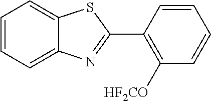

The reaction was performed according to the general procedure for the difluoromethylation of phenols on a 0.5 mmol scale. The product was purified by silica gel chromatography to give 2r as a white solid (107 mg, 77% yield).

¹H NMR (600 MHz, CDCl₃) δ 8.54 (d, J=7.9 Hz, 1H), 8.16 (d, J=8.1 Hz, 1H), 7.96 (d, J=8.0 Hz, 1H), 7.53 (dt, J=12.3, 7.9 Hz, 2H), 7.44 (t, J=7.6 Hz, 1H), 7.40 (t, J=7.6 Hz, 1H), 7.31 (d, J=8.2 Hz, 1H), 6.71 (t, J=73.2 Hz, 1H).

¹³C NMR (151 MHz, CDCl₃) δ 161.96 (s), 152.14 (s), 148.78 (t, J=2.5 Hz), 135.92 (s), 131.77 (s), 130.62 (s), 126.33 (s), 125.92 (s), 125.44 (s), 125.38 (s), 123.21 (s), 121.41 (s), 119.54 (s), 116.12 (t, J=261.9 Hz).

¹⁹F NMR (376 MHz, CDCl₃) δ −82.68 (d, J=73.2 Hz).

### 4-(difluoromethoxy)-2H-chromen-2-one (2s)

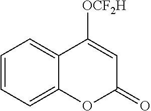

The reaction was performed according to the general procedure for the difluoromethylation of phenols on a 0.5 mmol scale. The product was purified by silica gel chromatography to give 2s (94 mg, 89% yield).

¹H NMR (400 MHz, CDCl₃) δ 7.82 (d, J=7.8 Hz, 1H), 7.63 (t, J=7.4 Hz, 1H), 7.36 (m, 2H), 6.81 (t, J=71.2 Hz, 1H), 5.97 (s, 1H).

¹³C NMR (151 MHz, CDCl₃) δ 160.96 (s), 158.93 (t, J=3.2 Hz), 153.52 (s), 133.40 (s), 124.53 (s), 123.00 (s), 114.41 (t, J=264.8 Hz), 114.05 (s), 96.43 (s).

¹⁹F NMR (376 MHz, CDCl₃) δ −87.63 (d, J=71.2 Hz).

### 3-(difluoromethoxy)-2-phenyl-4H-chromen-4-one (2t)

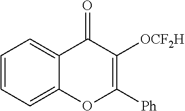

The reaction was performed according to the general procedure for the difluoromethylation of phenols on a 0.5 mmol scale. The product was purified by silica gel chromatography to give 2t as a white solid (114 mg, 79% yield).

¹H NMR (400 MHz, CDCl₃) δ 8.27 (d, J=8.0 Hz, 1H), 8.10-8.04 (m, 2H), 7.75 (t, J=7.8 Hz, 1H), 7.62-7.51 (m, 4H), 7.47 (t, J=7.6 Hz, 1H), 7.20 (t, J=76.9 Hz, 1H).

¹³C NMR (151 MHz, CDCl₃) δ 173.32 (s), 157.42 (s), 155.35 (s), 134.21 (s), 133.68 (t, J=4.0 Hz), 131.46 (s), 129.67 (s), 128.94 (s), 128.56 (s), 125.84 (s), 125.37 (s), 123.74 (s), 118.12 (s), 115.65 (t, J=262.6 Hz).

¹⁹F NMR (376 MHz, CDCl₃) δ −84.38 (d, J=76.9 Hz).

### Difluoromethyl-capsaicin (2w)

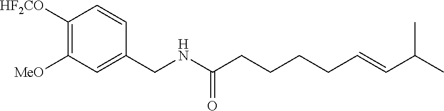

Note: Commercially available capsaicin from natural sources (TCI Chemicals) is a 1.9:1 mixture of capsaicin and dihydrocapsaicin. The mixture of capsaicin and dihydrocapsaicin was used as received. The reaction was performed according to the general procedure for the difluoromethylation of phenols on a 0.5 mmol scale. The product was purified by silica gel chromatography to give 2w as a white solid (80 mg, 45% yield) as an inseparable 1.9:1 mixture of difluoromethyl-capsaicin and difluoromethyl-dihydrocapsaicin.

### Difluoromethyl-Capsaicin:

¹H NMR (600 MHz, CDCl₃) δ 7.09 (d, J=8.1 Hz, 1H), 6.89 (s, 1H), 6.81 (d, J=8.1 Hz, 1H), 6.51 (t, J=75.2 Hz, 1H), 5.84 (s, 1H), 5.46-5.23 (m, 2H), 4.40 (d, J=5.8 Hz, 2H), 3.85 (s, 3H), 2.21 (t, J=7.5 Hz, 2H), 1.65 (dd, J=15.1, 7.6 Hz, 2H), 1.43-1.34 (m, 2H), 1.34-1.22 (m, 3H), 0.94 (d, J=6.7 Hz, 6H).

¹³C NMR (151 MHz, CDCl₃) δ 173.03 (s), 151.13 (s), 139.04 (t, J=3.0 Hz), 138.05 (s), 137.42 (s), 126.34 (s), 122.30 (s), 119.83 (s), 116.07 (t, J=259.8 Hz), 112.13 (s), 55.86 (s), 43.05 (s), 36.46 (s), 32.13 (s), 30.87 (s), 29.20 (s), 25.19 (s), 22.54 (s).

¹⁹F NMR (376 MHz, CDCl₃) δ −82.92 (d, J=75.2 Hz).

### Difluoromethyl-Dihydrocapsaicin:

¹H NMR (600 MHz, CDCl₃) δ 7.09 (d, J=8.1 Hz, 1H), 6.89 (s, 1H), 6.81 (d, J=8.1 Hz, 1H), 6.51 (t, J=75.2 Hz, 1H), 5.84 (s, 1H), 4.40 (d, J=5.8 Hz, 2H), 3.85 (s, 3H), 1.98 (dd, J=14.1, 7.0 Hz, 2H), 1.65 (dd, J=15.1, 7.6 Hz, 2H), 1.54-1.45 (m, 1H), 1.35-1.20 (m, 8H), 0.85 (d, J=6.6 Hz, 6H).

¹³C NMR (151 MHz, CDCl₃) δ 173.15 (s), 151.13 (s), 139.04 (t, J=3.0 Hz), 137.44 (s), 122.30 (s), 119.83 (s), 116.07 (t, J=259.8 Hz), 112.13 (s), 55.86 (s), 43.05 (s), 38.87 (s), 36.63 (s), 29.54 (s), 29.29 (s), 27.85 (s), 27.16 (s), 25.72 (s), 22.52 (s).

¹⁹F NMR (376 MHz, CDCl₃) δ −82.92 (d, J=75.2 Hz).

### Difluoromethyl-estrone (2x)

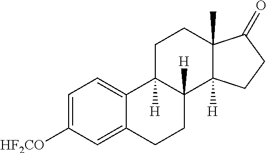

The reaction was performed according to the general procedure for the difluoromethylation of phenols on a 0.5 mmol scale. The product was purified by silica gel chromatography to give 2x (81 mg, 50% yield).

¹H NMR (600 MHz, CDCl₃) δ 7.27 (d, J=8.4 Hz, 1H), 6.91 (d, J=8.6 Hz, 1H), 6.86 (s, 1H), 6.47 (t, J=74.3 Hz, 1H), 2.91 (dd, J=8.7, 3.6 Hz, 2H), 2.51 (dd, J=19.1, 8.8 Hz, 1H), 2.43-2.38 (m, 1H), 2.27 (t, J=10.8 Hz, 1H), 2.05 (ddd, J=23.9, 13.4, 5.9 Hz, 2H), 1.97 (d, J=10.5 Hz, 1H), 1.69-1.40 (m, 7H), 0.91 (s, 3H).

¹³C NMR (151 MHz, CDCl₃) δ 220.57 (s), 149.20 (t, J=2.6 Hz), 138.46 (s), 137.01 (s), 126.71 (s), 119.68 (s), 116.86 (s), 116.07 (t, J=259.1 Hz), 50.42 (s), 47.91 (s), 44.05 (s), 38.06 (s), 35.82 (s), 31.54 (s), 29.43 (s), 26.29 (s), 25.79 (s), 21.57 (s), 13.81 (s).

¹⁹F NMR (376 MHz, CDCl₃) δ −82.49 (d, J=74.3 Hz).

### Example 2

### One-Pot Difluoromethoxylation of Arylboronic Acids

TABLE 3
One-pot difluoromethoxylation of arylboronic acids.

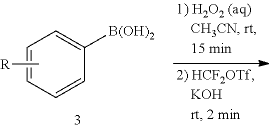

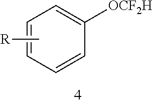

| | |
|---|---|
| 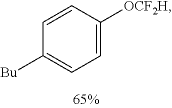 | 4a |
| 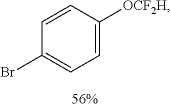 | 4b |
| 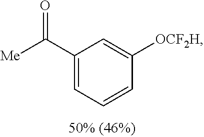 | 4c |
| 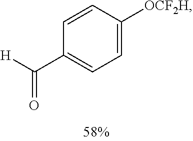 | 4d |
| 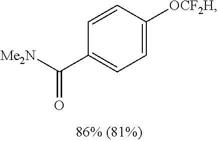 | 4e |

^{[a]}Reactions were performed on a 0.1 mmol scale to determine yields by ¹⁹F NMR spectroscopy with PhCF₃ as an internal standard added after the reaction. Isolated yields are shown in parenthesis for reactions performed on a 0.5 mmol scale.

### General Procedure for the Difluoromethoxylation of Aryl Boronic Acids

To a 20 mL vial was added the aryl boronic acid (0.5 mmol, 1.0 equiv), acetonitrile (1.0 mL) and 30% aqueous hydrogen peroxide (500 μL). The reaction was stirred at room temperature for 15 minutes. After this time, 12M KOH (500 μL) was added carefully. Note: the addition of KOH causes rapid decomposition of the unreacted hydrogen peroxide. This reaction is exothermic, and gas is evolved. The resulting mixture was stirred rapidly at room temperature, and HCF₂OTf (210 μL, 1.5 mmol, 3.0 equiv) was added at once. Note: the reactions are exothermic. The mixture was stirred vigorously for 2 minutes. The reaction was diluted with H₂O (8 mL) and extracted with ether (2×8 mL). The combined organic layers were dried over MgSO₄, concentrated, and purified by silica gel chromatography.

### 3′-(difluoromethoxy)acetophenone (4c)

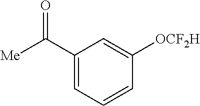

The reaction was performed according to the general procedure for the difluoromethoxylation of boronic acids on a 0.5 mmol scale. The product was purified by silica gel chromatography to give 4c as a clear oil (43 mg, 46% yield).

¹H NMR (600 MHz, CDCl₃) δ 7.80 (d, J=7.7 Hz, 1H), 7.70 (s, 1H), 7.48 (t, J=7.8 Hz, 1H), 7.33 (d, J=8.1 Hz, 1H), 6.57 (t, J=73.3 Hz, 1H), 2.61 (s, 3H).

¹³C NMR (151 MHz, CDCl₃) δ 196.80 (s), 151.26 (t, J=2.8 Hz), 138.79 (s), 130.06 (s), 125.32 (s), 124.26 (s), 118.93 (s), 115.60 (t, J=261.1 Hz), 26.59 (s).

¹⁹F NMR (376 MHz, CDCl₃) δ −83.61 (d, J=73.3 Hz).

### 4-(difluoromethoxy)-N,N-dimethylbenzamide (4e)

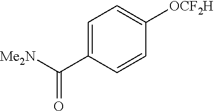

The reaction was performed according to the general procedure for the difluoromethoxylation of boronic acids on a 0.5 mmol scale. The product was purified by silica gel chromatography to give 4e as a white solid (87 mg, 81% yield).

¹H NMR (600 MHz, CDCl₃) δ 7.45 (d, J=8.2 Hz, 2H), 7.15 (d, J=8.1 Hz, 2H), 6.54 (t, J=73.4 Hz, 1H), 3.10 (s, 3H), 3.01 (s, 3H).

¹³C NMR (151 MHz, CDCl₃) δ 170.62 (s), 151.89 (t, J=2.8 Hz), 133.41 (s), 129.01 (s), 119.25 (s), 115.61 (t, J=260.7 Hz), 39.50 (br, s), 35.46 (br, s).

¹⁹F NMR (376 MHz, CDCl₃) δ −83.51 (d, J=73.4 Hz).

### Example 3

### One-Pot Difluoromethoxylation of Aryl Halides

TABLE 4
One-pot difluoromethoxylation of aryl halides

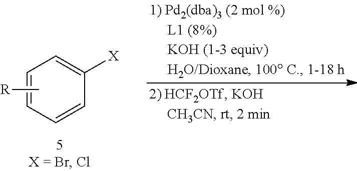

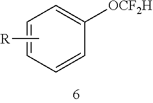

| | |
|---|---|
| 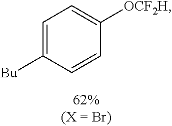 | 6a |
| 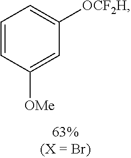 | 6b |
| 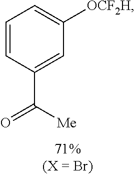 | 6c |
| 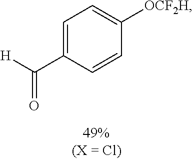 | 6d |
| 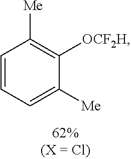 | 6e |
| 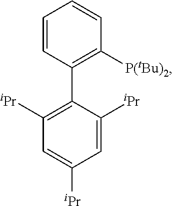 | L1 |

^{[a]}Reactions were performed on a 0.5 mmol scale to determine yields by ¹⁹F NMR spectroscopy with PhCF₃ as an internal standard added after the reaction.

### General Procedure for the Difluoromethoxylation of Aryl Bromides and Aryl Chlorides

Note: The hydroxylation reaction was set-up under an inert atmosphere according to the literature procedure. [Anderson, K. W.; Ikawa, T.; Tundel, R. E.; Buchwald, S. L. J. Am. Chem. Soc. 2006, 128, 10694.] To an oven-dried 4 mL vial was added Pd₂(dba)₃ (9.2 mg, 0.010 mmol, 4.0 mol % Pd), 2-Di-tert-butylphosphino-2′,4′,6′-triisopropylbiphenyl (^{t}Bu-XPhos, 17.0 mg, 0.040 mmol, 8.0 mol %), KOH (1.0-3.0 equiv), degassed H₂O (150-300 μL) and dioxane (250-500 μL). The aryl halide (0.5 mmol, 1.0 equiv) was added (solid aryl halides were weighed into the vial prior to adding solvent, and liquid aryl bromides were added neat by syringe after the addition of solvent). The vial was sealed with a Teflon-lined cap and heated at 100° C. for 1-18 h. The solution was allowed to cool, and the reaction was diluted with acetonitrile (500-750 μL, such that the total volume of dioxane and acetonitrile is 1.0 mL) and 6M KOH (700-850 μL, such that the final aqueous solvent volume is 1.0 mL). The resulting mixture was stirred rapidly at room temperature, and HCF₂OTf (210 μL, 1.5 mmol, 3.0 equiv) was added at once. Note: the reactions are exothermic. The mixture was stirred vigorously for 2 minutes. The reaction was diluted with H₂O (8 mL) and extracted with ether (2×8 mL). The combined organic layers were dried over MgSO₄, concentrated, and purified by silica gel chromatography.

### Example 4

### One-Pot Difluoromethoxylation of Arenes Through Ir-Catalyzed C—H Borylation

TABLE 5
One-pot difluoromethoxylation of arenes through Ir-catalyzed
C—H borylation.

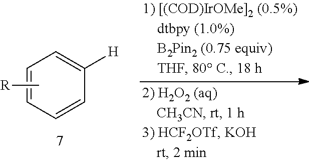

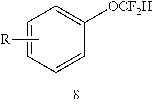

| | |
|---|---|
| 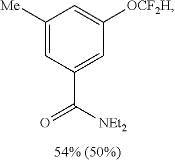 | 8a |
| 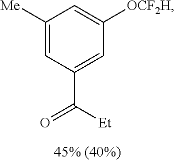 | 8b |
| 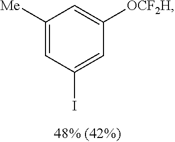 | 8c |
| 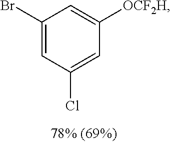 | 8d |
| 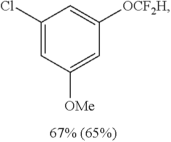 | 8e |

^{[a]}Reactions were performed on a 0.1 mmol scale to determine yields by ¹⁹F NMR spectroscopy with PhCF₃ as an internal standard added after the reaction. Isolated yields are shown in parenthesis for reactions performed on a 0.5 mmol scale.

### General Procedure for the Difluoromethoxylation of Arenes Through Ir-Catalyzed C—H Borylation

Note: The borylation reaction was set-up under an inert atmosphere. To an oven-dried 20 mL vial was added arene (0.5 mmol, 1.0 equiv), and 1.0 mL of a stock solution containing 0.5 mol % [Ir(COD)OMe]₂, 1.0 mol % 4,4′-di-tert-butyl bipyridine (dtbpy), and 0.75 equiv of B₂Pin₂. The vial was sealed with a Teflon-lined cap and heated at 80° C. for 18 h. The solution was allowed to cool, and the volatile components were removed in vacuo. To the crude ArBPin was added acetonitrile (1.0 mL) and 30% aqueous hydrogen peroxide (500 μL). The reaction was stirred at room temperature for 15 minutes. After this time, 12M KOH (500 μL) was added carefully. Note: the addition of KOH causes rapid decomposition of the unreacted hydrogen peroxide. This reaction is exothermic, and gas is evolved. The resulting mixture was stirred rapidly at room temperature, and HCF₂OTf (210 μL, 1.5 mmol, 3.0 equiv) was added at once. Note: the reactions are exothermic. The mixture was stirred vigorously for 2 minutes. The reaction was diluted with H₂O (8 mL) and extracted with ether (2×8 mL). The combined organic layers were dried over MgSO₄, concentrated, and purified by silica gel chromatography.

### 3-(difluoromethoxy)-N,N-diethyl-5-methylbenzamide (8a)

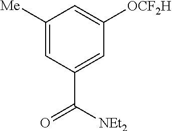

The reaction was performed according to the general procedure for the difluoromethoxylation of arenes on a 0.5 mmol scale. The product was purified by silica gel chromatography to give 8a as a white solid (64 mg, 50% yield).

¹H NMR (600 MHz, CDCl₃) δ 7.01 (s, 1H), 6.94 (s, 1H), 6.91 (s, 1H), 6.49 (t, J=73.8 Hz, 1H), 3.52 (d, J=5.6 Hz, 2H), 3.22 (d, J=5.5 Hz, 2H), 2.36 (s, 3H), 1.23 (s, 3H), 1.10 (s, 3H).

¹³C NMR (151 MHz, CDCl₃) δ 169.99 (s), 150.94 (t, J=2.7 Hz), 140.63 (s), 138.78 (s), 123.92 (s), 120.74 (s), 115.77 (t, J=260.0 Hz), 114.34 (s), 43.23 (s), 39.25 (s), 21.27 (s), 14.10 (s), 12.79 (s).

¹⁹F NMR (376 MHz, CDCl₃) δ −83.16 (d, J=73.8 Hz).

### 1-(3-(difluoromethoxy)-5-methylphenyl)propan-1-one (8b)

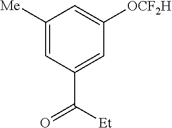

The reaction was performed according to the general procedure for the difluoromethoxylation of arenes on a 0.5 mmol scale. The product was purified by silica gel chromatography to give 8b (43 mg, 40% yield).

¹H NMR (600 MHz, CDCl₃) δ 7.62 (s, 1H), 7.50 (s, 1H), 7.13 (s, 1H), 6.54 (t, J=73.6 Hz, 1H), 2.98 (q, J=7.1 Hz, 2H), 2.42 (s, 3H), 1.22 (t, J=7.2 Hz, 3H).

¹³C NMR (151 MHz, CDCl₃) δ 199.79 (s), 151.25 (t, J=2.8 Hz), 140.51 (s), 138.47 (s), 125.66 (s), 124.61 (s), 115.90 (s), 115.72 (t, J=260.5 Hz), 31.95 (s), 21.31 (s), 8.12 (s).

¹⁹F NMR (376 MHz, CDCl₃) δ −83.37 (d, J=73.6 Hz).

### 1-(difluoromethoxy)-3-iodo-5-methylbenzene (8c)

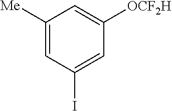

The reaction was performed according to the general procedure for the difluoromethoxylation of arenes on a 0.5 mmol scale. The product was purified by silica gel chromatography to give 8c (60 mg, 42% yield).

¹H NMR (600 MHz, CDCl₃) δ 7.40 (s, 1H), 7.29 (s, 1H), 6.90 (s, 1H), 6.47 (t, J=73.5 Hz, 1H), 2.32 (s, 3H).

¹³C NMR (151 MHz, CDCl₃) δ 151.20 (t, J=2.9 Hz), 141.81 (s), 135.29 (s), 125.71 (s), 119.86 (s), 115.63 (t, J=260.7 Hz), 93.72 (s), 20.91 (s).

¹⁹F NMR (376 MHz, CDCl₃) δ −83.44 (d, J=73.5 Hz).

### 1-bromo-3-chloro-5-(difluoromethoxy)benzene (8d)

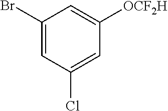

The reaction was performed according to the general procedure for the difluoromethoxylation of arenes on a 0.5 mmol scale. The product was purified by silica gel chromatography to give 8d as a clear oil (89 mg, 69% yield).

¹H NMR (600 MHz, CDCl₃) δ 7.38 (s, 1H), 7.21 (s, 1H), 7.10 (s, 1H), 6.50 (t, J=72.6 Hz, 1H).

¹³C NMR (151 MHz, CDCl₃) δ 151.57 (t, J=2.7 Hz), 135.88 (s), 128.63 (s), 122.99 (s), 121.49 (s), 119.17 (s), 115.22 (t, J=263.2 Hz).

¹⁹F NMR (376 MHz, CDCl₃) δ −84.55 (d, J=72.6 Hz).

### 1-chloro-3-(difluoromethoxy)-5-methoxybenzene (8e)

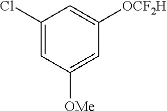

The reaction was performed according to the general procedure for the difluoromethoxylation of arenes on a 0.5 mmol scale. The product was purified by silica gel chromatography to give 8e as a clear oil (68 mg, 65% yield).

¹H NMR (600 MHz, CDCl₃) δ 6.74 (d, J=14.0 Hz, 1H), 6.56 (s, 1H), 6.49 (t, J=73.4 Hz, 1H), 3.79 (s, 3H).

¹³C NMR (151 MHz, CDCl₃) δ 161.08 (s), 152.32 (t, J=3.0 Hz), 135.54 (s), 115.59 (t, J=260.8 Hz), 112.06 (s), 111.51 (s), 104.40 (s), 55.72 (s).

¹⁹F NMR (376 MHz, CDCl₃) δ −83.90 (d, J=73.4 Hz).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition comprising:
(i) a precursor compound selected from an aryl alcohol, aryl thioalcohol and a vinylic alcohol, any of which are optionally further substituted;
(ii) a difluoromethyl source having the formula HCF₂OSO₂R^{x},
wherein R^{x} is fluoroalkyl or aryl;
(iii) a base; and
(iv) water.

2. The composition according to claim 1, further comprising a co-solvent.

3. The composition according to claim 2, wherein said co-solvent is a member selected from DMF, DMSO, dioxane, THF, and MeCN.

4. The composition according to claim 1, wherein R^{x} is a member selected from C₁, C₂, C₃, C₄, C₅ and C₆ perfluoroalkyl.

5. The composition according to claim 1, wherein said difluoromethyl source is difluoromethyltriflate (HCF₂Tf).

6. The composition according to claim 1, wherein said difluoromethyl source is difluoromethylnonaflate (HCF₂Nf).

7. The reaction mixture according to claim 1, wherein said base is a member selected from KOH, LiOH, and NaOH.

8. The composition according to claim 1, wherein said precursor compound, said difluoromethyl source, and said base are present in said mixture in a molar ratio of about 1:3:12.

9. The composition according to claim 1, wherein said precursor compound has the formula: 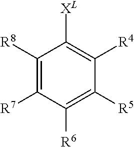
wherein
R⁴, R⁵, R⁶, R⁷, and R⁸ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂, wherein two or more of R⁴, R⁵, R⁶, R⁷ and R⁸, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl,
wherein
R⁹ and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl; and
X^{L} is a member selected from —OH, —SH, —O⁻ and —S⁻.

10. The composition according to claim 1, wherein said precursor compound is an aryl alcohol or aryl thioalcohol; and said precursor compound was synthesized in situ.

11. The composition according to claim 1, wherein said aryl alcohol was synthesized in situ from an arylboronic acid, an aryl halide, or an arene.

12. The composition according to claim 1, wherein said precursor compound has the formula: 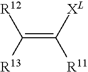
wherein
R¹¹, R¹², and R¹³ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂, wherein two or more of R¹¹, R¹², and R¹³, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl,
wherein
R⁹ and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring; and
X^{L} is —OH or —O⁻.

13. A method for forming a difluoromethyl ether or a difluoromethyl sulfide, said method comprising:
(a) forming a composition according to claim 1; and
(b) incubating said composition under conditions appropriate to form said difluoromethyl ether or said difluoromethyl sulfide by difluoromethylating the alcohol or thioalcohol moiety of the precursor compound.

14. The method according to claim 13, wherein said reaction mixture is incubated at a temperature from about 0° C. to about 40° C.

15. The method according to claim 14, wherein said reaction mixture is incubated at room temperature (about 20° C.).

16. The method according to claim 13, wherein said mixture is incubated in a sealed tube.

17. The method according to claim 13, wherein said mixture is incubated for about 1 minute to about 24 hours.

18. The method according to claim 17, wherein said mixture is incubated for about 1 minute to about 1 hour.
